(19) United States
(12) Patent Application Publication
(10) Pub. No.: US 2010/0087399 A1
(43) Pub. Date: Apr. 8, 2010

(54) USE OF 9-OXOACRIDINE-10-ACETIC ACID, SALTS AND ESTERS THEREOF IN COMBINATION THERAPY OF OVARIAN CANCER

(76) Inventor: Kirill Gennadievich SURKOV, Saint Petersburg (RU)

Correspondence Address: PATENTICA LLC, 51 Sadovaya Street, Office 303, Saint Petersburg, 190068, RU

(21) Appl. No.: 12/569,112

(22) Filed: Sep. 29, 2009

Related U.S. Application Data
(63) Continuation-in-part of application No. PCT/RU2008/000205, filed on Mar. 31, 2008.

(30) Foreign Application Priority Data
Mar. 29, 2007 (RU) 2007111680

(51) Int. Cl. A61K 31/675; A61K 31/473; A61P 35/00

(52) U.S. Cl. 514/90; 514/297

(57) ABSTRACT

The present invention provides novel methods of combination therapy of ovarian cancer, pharmaceutical kits and combinations of 9-oxoacridine-10-acetic acid and/or salts and/or esters thereof with one or more chemotherapeutic agents. The proposed combination therapy is useful in enhancing the action of chemotherapeutic agents and their proliferative activity on human ovarian cancer cells.

### CROSS-REFERENCE TO RELATED APPLICATIONS

This application claims the benefits from earlier filed applications PCT/RU2008/000205 of Mar. 31, 2008 and RU2007111680 of Mar. 29, 2007. The contents of these applications are hereby incorporated by reference and in their entirety.

### FIELD OF THE INVENTION

The present invention relates to combination therapy of ovarian cancer using 9-oxoacridine-10-acetic acid and/or salts and esters thereof in combination with one or more chemotherapeutic agents.

### BACKGROUND OF THE INVENTION

Ovarian cancer (OC) is developed from the cells of hormone-dependent tissue, and estrogen and progestin receptors are present in the cytosol of these tumor cells.

However, attempts to use progestins, antiesrtrogens and aromatase inhibitors showed no significant clinical efficacy (see Shuk-Mei Ho, Estrogen, Progesterone and Epithelial Ovarian Cancer, Reproductive Biology and Endocrinology 2003; vol. 1, p. 73-80). Nowadays, chemotherapy still remains one of major methods of cancer therapy. The intensive research continues in an effort to find new agents that are capable of enhancing the efficacy of ovarian cancer chemotherapy.

The use of immunomodulators in therapy of advanced ovarian cancer, as well as in therapy of other cancers is well know.

One group of cytokine immunomodulators comprises interferon and its derivatives, which possess antiviral and antitumor activity. For instance, U.S. Pat. No. 5,268,169 discloses a method of ovarian cancer therapy using gamma-interferon intraperitoneal perfusion.

The use of complex platinum compounds, taxans and gamma-interferon in combination therapy of ovarian cancer is also known, see e.g. US publication US2004/0191218.

Further, it has been reported that the addition of gamma-interferon to polychemotherapy of OC (cisplatinum+cyclophosphamide) increases from 38% to 51% the number of patients who had no progression within the 3 year period (see: A randomized phase III trial of cisplatin/cyclophosphamide plus or minus interferon—gamma in the first-line therapy of ovarian cancer: update analysis. Program and abstracts of the 31st Annual Meeting of the Society of Gynecologic Oncologists; Feb. 5-9, 2000; San Diego, Calif. Abstract 2).

As a treatment for OC, the intraperitoneal administration of alpha-interferon in combination with carboplatinum is proposed (Frasci, G., Tortoriello, A., et al. Carboplatin and alpha-2b interferon intraperitoneal combination as first-line treatment of minimal residual ovarian cancer. A pilot study, European Journal of Cancer, 1994; vol. 30A, pp. 946-50).

However, this therapy did not prove any efficacy in respect of ovarian cancer. (M. Bruzzone, A. Rubagotti et al., Intraperitoneal Carboplatin with or without Interferon-a in Advanced Ovarian Cancer Patients with Minimal Residual Disease at Second Look: A Prospective Randomized Trial of 111 Patients. Gynecologic Oncology, 1997, vol. 65, pp. 499-505). Since rather high interferon doses shall be administered intraperitoneally to achieve noticeable response of OC in clinical trials, this method is hardly applicable because of significant side effects of interferon. Thus, there is still the necessity of increasing the efficacy of OC treatment as well as the unmet need in more efficient methods of treating OC.

Acridine derivatives, for example, 9-oxoacridine-10-acetic acid and its salts have been widely used as immunomodulators in various medical applications.

Moreover, some acridine derivatives are reported to inhibit telomerase activity and show own anti-tumor properties. For example, the use of acridine derivatives for enhancement efficacy of anti-tumor agents is disclosed in U.S. Pat. No. 5,604,237.

9-oxoacridine-10-acetic acid has the following structural formula:

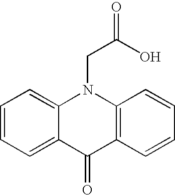

According to another nomenclature, the name of this compound is 10-(carboxymethyl)-9(10H)acridone, the CAS number is 38609-97-1, and the international nonproprietary name is cridanimod.

It will be appreciated that within the description of the present application, the abbreviation “CMA” designates 9-oxoacridine-10-acetic acid itself as well as pharmaceutically acceptable salts and esters thereof if otherwise is not explicitly indicated or other meaning is not readily apparent from the context.

The use of 9-oxoacridine-10-acetic acid derivatives have been first disclosed as powerful antiviral agents by Hoffman La Rosh Inc. employees (see U.S. Pat. No. 3,681,360)

Nowadays, medicaments on the basis of 9-oxoacridine-10-acetic acid and its pharmaceutically acceptable salts are used for the treatment and prevention of a wide range of diseases. In particular, its immunomodulatory, interferonogenous, antibacterial, anti-promoter and radioprotective properties are well known.

The nuclear factor kappa B (NF κB) family is composed of specific cytoplasmatic proteins activated in eukaryote cells by negative influences, particularly by chemotherapy and radiation. Non-active form of this protein is present in a complex with its own inhibitor. Once activated, (by specific phosphorylkinases) the complex decomposes and protein NF κB is translocated into the nucleus and switches on the target genes.

The activation of these genes is associated with proliferation, angiogenesis, apoptosis suppression which are key links in development of resistance of tumor cells to chemotherapy.

The agents inhibiting NF κB activity may act on any step of activation of the factor; e.g. they can bind NF κB; inhibit translocation NF κB into the nucleus; inhibit regulation of YY transcription factor with NF κB; inhibit apoptosis promotion; inhibit formation of complex of NF κB with other factors regulating NF κB activity.

The search for new NF κB inhibitors is carried out by wide front, including screening among known compounds. For example, dehydroxymethylepoxyquinomycin (DHMEQ) (a structural analog of antibiotic epoxyquinomycin C and its derivatives) (see WO 2006/060819), curcumin, its derivatives (see WO 03/090618) and other compounds possess the ability to inhibit NF κB activity.

However, while various investigators have been studying the ways to enhance the efficacy of cancer therapy, none have proposed the combination therapy of this invention. The objective of present invention is to provide the improved treatment of ovarian cancer.

### BRIEF SUMMARY OF THE INVENTION

The inventors of the present invention have now surprisingly found that the use of 9-oxoacridine-10-acetic acid and/or pharmaceutically acceptable salts and esters thereof significantly increases the action of chemotherapeutic agents on ovarian cancer cells.

Though 9-oxoacridine-10-acetic acid alone possesses no noticeable cytostatic activity, it has been surprisingly demonstrated in the present invention that in the presence of 9-oxoacridine-10-acetic acid and/or its salts inhibition of ovarian cancer call growth with conventional chemotherapy is much more effective than the same chemotherapy in the absence of CMA.

It is known that 9-oxoacridine-10-acetic acid has interferon-inducing properties. However, in cell culture studies, the inventors of the present invention observed no measurable increase of interferon level, and thus, it shall be concluded that the newly-discovered property of CMA does not results from interferon activity and is not mediated by interferon.

Further, in vivo studies of ovarian malignant tumors growth inhibition by the inventors of the present invention have demonstrated that 9-oxoacridine-10-acetic acid continues to reveal dose-dependent effect at doses which exceed maximal interferon-inducing dose, i.e. the threshold dose of 9-oxoacridine-10-acetic acid, further increase of which does not lead to increased tissue and serum interferons levels.

Moreover, in case of depletion of interferon system (which is typically observed upon repeated administration of any interferon inducer including 9-oxoacridine-10-acetic acid) 9-oxoacridine-10-acetic acid and/or its pharmaceutically accepted salts and its esters continue to reveal dose-dependent influence on the tumor growth inhibition when combined with chemotherapy

Without being bound by theory, it is believed that this inhibition can be caused by either decrease in the cell proliferative activity, or accelerated cell apoptosis, or both.

Thus, a new effect of 9-oxoacridine-10-acetic acid has been found by the inventors of the present invention though at present the mechanism of this effect is not entirely clear. However, realization of this mechanism leads to a decrease of active NF κB level and opens new frontiers in use of 9-oxoacridine-10-acetic acid and/or its pharmaceutically accepted salts and esters.

As has been found by the inventors of the present invention, 9-oxoacridine-10-acetic acid and/or its salts and esters thereof inhibits NF κB activity in ovarian cancer cells. Due to this effect, when chemotherapeutic agents are applied subsequently or in parallel with CMA, their cytostatic or cytolytic action on ovarian cancer cells enhance markedly.

The chemotherapeutic agents according to the invention can be selected without limitation, from the following: complex platinum compounds (in particular, cisplatin, carboplatin, oxalyplatin); antimetabolites (in particular, methotrexate, 5-fluorouracil, fluorafur, 6-mercaptopurin, altretamine, gemcitabine); alkylating agents (in particular, cyclophosphamide, chlorambucil, melphalan); antitumor antibiotics (in particular, doxorubicin, epirubicin, mitoxantrone); taxans (in particular, paclitaxel, docetaxel); topoisomerase I inhibitors (in particular, topotecan, irinotecan).

Based of these newly-discovered CMA properties, according to the present invention, a novel method of ovarian cancer therapy using 9-oxoacridine-10-acetic acid and/or salts and/or esters thereof is provided, the method comprising the use of 9-oxoacridine-10-acetic acid and/or salts and/or esters thereof in combination with one or more chemotherapeutic agents, thereby increasing the efficacy of chemotherapy greatly.

Thus, in one aspect of the present invention, a method of ovarian cancer treatment is provided, the method comprising administering9-oxoacridine-10-acetic acid and/or salts and/or esters thereof in combination with one or more chemotherapeutic agents, wherein 9-oxoacridine-10-acetic acid and/or salts and/or esters thereof is administered in amounts effective in potentiating the action of the said one or more chemotherapeutic agents.

Preferable regimens of ovarian cancer treatment using CMA, salts and esters thereof are described in more detail below.

In further embodiments of the present invention, variants of the inventive method with the use of chemotherapeutic agents of different classes are disclosed.

According to the present invention, preferred classes of chemotherapeutic agents as well as their representatives include, but not limited to: complex platinum compounds, such as cisplatin, carboplatin, oxalyplatin; antimetabolites, such as methotrexate, 5-fluorouracil, fluorafur, 6-mercaptopurin, altretamine, gemcitabine; alkylating agents, such as cyclophosphamide, chlorambucil, melphalan; antitumor antibiotics, such as doxorubicin, epirubicin, mitoxantrone; taxans, such as paclitaxel, docetaxel; topoisomerase I inhibitors, such as topotecan, irinotecan.

Further, according to the invention, variants of the inventive method have been studied in clinical practice using chemotherapeutic agents of different classes, and the experimental results are provided below. It shall be also appreciated that a method of combined therapy according to the invention can be used as a (i) sole or (ii) combined ovarian cancer therapy. For example, a method according to the invention can be used as a part of a complex therapy, such as neoadjuvant or adjuvant treatment when tumor lesions are eliminated surgically.

It has also been found by the inventors of the present invention that CMA therapy when combined with additional hormonotherapy aimed to decrease estrogenic stimulation associated with the action of endogenous estrogens (both gonadal and extragonadal) results in even more significant inhibition of NF κB activity upon ovarian tumor cells.

Thus, according to a further embodiment of the present invention, a method of treating ovarian cancer in a subject in need thereof is provided, wherein the method comprises (a) administering 9-oxoacridine-10-acetic acid and/or salts and/or esters thereof in a combination therapy with one or more chemotherapeutic agents, and (b) hormonotherapy aimed to decrease the effect of endogenous estrogens.

The term [symbol] estrogenic action [symbol] refers to any action associated with cytosol estrogen receptors stimulation, which results inactivation of estrogen-induced genes in the cell.

According to the invention, the said decrease of estrogen action can be achieved by any known method of hormonotherapy, such as estrogen receptor blockage (using, for example, antiestrogens), lowering estrogen receptor concentration (using, for example, progestins), reducing both gonadal and extragonadal conversion of androgens into estrogens (using, for example, aromatase inhibitors), or by suppressing gonadal synthesis of estrogens (using, for example, agonists and antagonists of luteinizing hormone-releasing hormone (LHRH)). Other means of hormonotherapy can be used as well.

Without being bound by theory, it is believed that LHRH agonists (for example, buserelin; goserelin) cause desensitization of hypophysis and alter its function, while LHRH antagonists (for example, cetrorelix, abarelix, ganirelix) in contrast to LHRH agonists, cause a complete blockade of hypophyseal membrane LHRH-receptors. In both cases, the production of follicle-stimulating hormone is decreased and it leads to inhibition of gonadal estrogen production and drop in blood estrogens level.

Thus, in a further embodiment of the method of treating ovarian cancer according to the present invention comprises (a) administering 9-oxoacridine-10-acetic acid and/or salts and/or esters thereof in a combination with (b) one or more chemotherapeutic agents and further, in a combination with (c) one or more hormonetherapeutic agents selected from the group including anti-estrogens, progestins, aromatase inhibitors, LHRH-antagonists, LHRH-agonist.

### DETAILED DESCRIPTION OF THE INVENTION

As used herein, the terms “to treat,” “treating,” and “treatment” refers to administering a therapy, an agent, a compound, or a composition to a subject in need of such a treatment, e.g. suffering from a disease or pathologic condition, wherein the subject can be a human, for example a patient or a person with risk of developing a pathology condition, such as ovarian cancer. In general, the treatment is provided to a subject having a disorder (for example, malignant tumor, in particular, ovarian cancer), a symptom or symptoms of a disorder, increased risk of a disorder, or a predisposition to a disorder, to cure, to recover, or to improve the life quality; to alleviate symptoms; to diminish the extent of disorder, or symptoms of a disorder, or a degree of predisposition to a disorder, to induce stabilization (i.e., not worsening) of the state of disorder, to delay or slow down progression of a disorder, to induce amelioration or palliation of the disorder state, and remission (either partial or total), either detectable or undetectable. The term “treatment” can also mean a prolonged survival as compared to expected survival in the absence of treatment. The term “treatment” can also mean the administration, introduction, prescription or applying otherwise a dose of a therapeutic agent, a composition, a compound alone or in combination with one or more other agents, compounds or compositions. As used herein, the terms “in combination”, “combination” refer to simultaneous or consecutive administration, introduction, prescription or applying otherwise a dose of various therapeutic agents, compositions, or compounds.

Further, the term “effective amount of a compound/drug” relates to an amount of this compound/drug effective to induce a specified action. The effective amount of one and the same compound/drug can vary depending on particular effect and particular combination, e.g. the amounts of CMA effective to induce the antitumor activity of cisplatine can differ from the amounts of CMA effective to induce the antitumor activity of paclitaxel. Similarly, the amounts of CMA effective to induce the antitumor activity of cisplatine can differ from the amounts of CMA effective to reduce the level of active NF KB factor.

None of the above mentioned newly-discovered properties of CMA, its salts and esters to alter NF κB activity and to enhance cytostatic effects of chemotherapeutic agents on ovarian cancer cells, as far as to the knowledge of the present inventors, have been disclosed in the prior art. Also, the inventors of the present invention are unaware of any mentioning or evidences of attempts to treat ovarian cancer using a combination of a chemotherapeutic agent with CMA or its salts or esters, and especially, to treat this disorder with a combination of CMA and chemotherapeutic agent when CMA is used in doses significantly exceeding the maximal interferon-inducing dose (14 mg/kg body weight and more).

The term “pharmaceutically acceptable salt” as used herein, means those salts, which maintain the above mentioned properties of 9-oxoacridine-10-acetic acid and which are not unacceptable biologically or unacceptable in some other way. The pharmaceutically acceptable salts derived from the salt forming bases could be obtained with inorganic or organic bases.

The salts with inorganic bases include, but are not limited to, sodium, potassium, lithium, ammonium, calcium and magnesium salts.

The salts with organic bases include, but are not limited to, salts of primary, secondary, tertiary and quaternary amines, such as alkylamines, dialkylamines, trialkylamines, substituted alkylamines, di(substituted alkyl)amines, tri(substituted alkyl)amines, alkenylamines, dialkenylamines, trialkenylamines, substituted alkenylamines, di(substituted alkenyl)amines, tri(substituted alkenyl)amines, cycloalkylamines, di(cycloalkyl)amines tri(cycloalkyl)amines, substituted cycloalkylamines, di(substituted cycloalkyl)amines, tri(substituted cycloalkyl)amines, cycloalkenylamines, di(substituted cycloalkenyl)amines, di(substituted cycloalkenyl)amines, arylamines, diarylamines, triarylamines, heteroarylamines, diheteroarylamines, triheteroarylamines, heterocyclylamines, diheterocyclylamines, triheterocyclylamines, mixed di- and tri-amines, where at least one of the substitutes on amine differs and is selected from the group, including alkyl, substituted alkyl, alkenyl, substituted alkenyl, aryl, heteroaryl, heterocyclyl, etc. Amines, in which two or three substitutes together with the nitrogen atom to which they are connected, form a heterocyclyl or a heteroaryl, also are included here.

Specific examples of appropriate amines include, in particular, isoprpylamine, trimethylamine, diethylamine, tri(isopropyl)amin, tri(n-propyl)amine, ethanolamine, 2-dimethylaminoethanol, tromethamine, lysine, arginine, histidine, caffeine, procaine, choline, betaine, ethylendiamine, glucosamine, N-alkylglucamine, theobromine, purines, piperazine, piperidine, morpholine, N-ethylpiperidine etc.

An example of a salt of 9-oxoacridine-10-acetic acid with an alkali metal is the sodium salt:

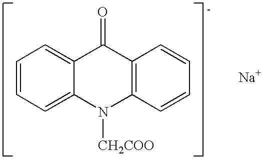

An example of a salt with amino compound is the salt with 1-deoxy-1-(methylamino)-D-glucitol (i.e. with meglumine, or, the same, with N-methylglucamine):

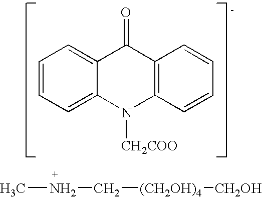

Other examples of salts with various complex quaternary ammonium bases include salts with amine-substituted carbohydrates, for example, with 2-deoxy-2-amino(or 2-alkylamino)-D-glucose, where R is H or a lower alkyl:

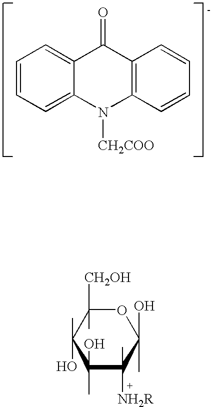

with 1-deoxy-1-methylamino-D-glucose:

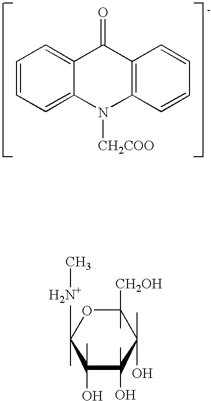

as well as salts with various esters of carbohydrates and aliphatic amino alcohols, for example

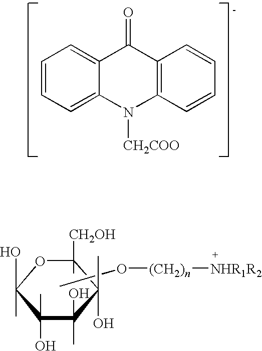

where R₁, R₂ are independently alkyl, aryl, heteryl

The examples of appropriate cations are, in particular, cations of 3-O—(N,N-dimethylamino-n-propyl)-1,2:5,6-di-O-isopropyliden-α,D-glucofuranose,

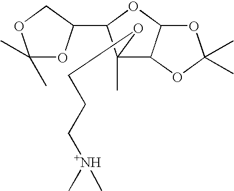

as well as following cations of:

1-deoxy-1-(ethylamino)-D-glucitol (i.e. eglumine),

1-deoxy-1-(propylamino)-D-glucitol,

1-deoxy-1-(butylamino)-D-glucitol,

1-deoxy-1-(methylamino)-L-glucitol,

1-deoxy-1-(ethylamino)-L-glucitol,

1-deoxy-1-(propylamino)-L-glucitol, and

1-deoxy-1-(butylamino)-L-glucitol.

According to the present specification, esters of 9-oxoacridine-10-acetic acid include compounds obtained by hydrogen atom substitution in acid OH-group with an organic group R.

Examples of suitable esters include but are not limited to, esters of 9-oxoacridine-10-acetic acid with lower alkyls (namely with (C₁-C₁₂)alkyls, in particular ethyl, propyl, isopropyl, butyl and amyl esters), as well as with choline and other lypophilic alcohols.

After rapid penetration through biological membranes in vivo, these compounds are easily hydrolyzed to free 9-oxoacridine-10-acetic acid.

According to the present invention, preferred salts of 9-oxoacridine-10-acetic acid are selected from the group including sodium, meglumine, eglumine salts and the salt with 3-O—(N,N-dimethylamino-n-propyl)-1,2:5 ,6-di-O-isopropyliden-α,D-glucofuranose.

Further, according to the present invention the use of 9-oxoacridine-10-acetic acid and/or its acceptable salts and/or its esters to produce a medication for ovarian cancer treatment in combination with a chemotherapeutic agent, is provided.

According to the present invention, a chemotherapeutic agent is preferably selected from the group including complex platinum compounds (in particular, cisplatin, carboplatin, oxalyplatin); antimetabolites (in particular, methotrexate, 5-fluorouracil, fluorafur, 6-mercaptopurin, altretamine, gemcitabine); alkylating agents (in particular, cyclophosphamide, chlorambucil, melphalan); antitumor antibiotics (in particular, doxorubicin, epirubicin, mitoxantrone); taxans (in particular, paclitaxel, docetaxel); topoisomerase I inhibitors (in particular, topotecan, irinotecan).

According to the present invention, preferred salts of 9-oxoacridine-10-acetic acid for production of a medication for ovarian cancer treatment in combination with a chemotherapeutic agents, are selected from the group including sodium, meglumine, eglumine salts and the salt with 3-O—(N,N-dimethylamino-n-propyl)-1,2:5,6-di-O-isopropyliden-α,D-glucofuranose.

Along with 9-oxoacridine-10-acetic acid and/or its acceptable salts and/or its esters to produce this inventive medication, other components can be used, including but not limited to: various excipients and additives, including: solubilizers, for example, aminosugars (or amino alcohols) and theirs esters, cyclodextrins, for example, hydroxypropyl-β-cyclodextrin; emulsifiers, for example, tweens; stiffeners; photo (light) filters, for example, methylene blue; complexing agents; stabilizers, for example, trilon B; prolongators, for example, methylcellulose and polyvinylpyrrolidone; corrigents, for example, sorbitol; dyes; preservatives; as well as organic- and non-organic buffer systems aimed to maintain a constant pH. This medication can be produced as a solution for injection, or as tablets, or as a enteric coated tablet, or as powder or a granulate in capsules or in sachets, or as suppositories, or as aerosol or solution for inhalation, or as prolonged form for oral use or form on the basis of solid or semi-solid a polymer matrix for abdominal implantation.

Further, the present invention provides a pharmaceutical kit for ovarian cancer treatment comprising 9-oxoacridine-10-acetic acid and/or its acceptable salts and/or its esters in a therapeutically effective amount and a chemotherapeutic agent.

In one embodiment, a pharmaceutical kit for ovarian cancer treatment in a patient in need of such treatment comprises a unit dosage of 9-oxoacridine-10-acetic acid and/or salt and/or ester thereof and a unit dosage of a chemotherapeutic agent, wherein 9-oxoacridine-10-acetic acid and/or salt and/or ester thereof is present in amount effective in reducing active NF kB level.

In another embodiment, a pharmaceutical kit for ovarian cancer treatment in a patient in need of such treatment comprises a unit dosage of 9-oxoacridine-10-acetic acid and/or salt and/or ester thereof and a unit dosage of a chemotherapeutic agent, wherein 9-oxoacridine-10-acetic acid and/or salt and/or ester thereof is present in amount effective for potentiating the action of the said chemotherapeutic agent or agents.

The above pharmaceutical kits can be made in the form of a single pharmaceutical pack including a separate blister of unit dosage(s) of 9-oxoacridine-10-acetic acid and/or salt and/or ester thereof and a separate blister of unit dosage(s) of a chemotherapeutic agent.

Alternatively, a kit can be made in the form of a two separate pharmaceutical packs including a pharmaceutical pack of unit dosage(s) of 9-oxoacridine-10-acetic acid and/or salt and/or ester thereof and a blister of unit dosage(s) of a chemotherapeutic agent.

In still another alternative, a kit can be made in the form of a combined blister comprising a unit dosage or dosages of 9-oxoacridine-10-acetic acid and/or salt and/or ester thereof and a unit dosage or dosages of a chemotherapeutic agent, in one blister, accompanied with the instruction on recommended administration regimens and dosages. According to the present invention, preferred salts of 9-oxoacridine-10-acetic acid in the kit for ovarian cancer treatment, are selected from the group including sodium, meglumine, eglumine salts and the salt with 3-O—(N,N-dimethylamino-n-propyl)-1,2:5,6-di-O-isopropyliden-α,D-glucofuranose.

According to the present invention, a chemotherapeutic agent in a kit for ovarian cancer treatment comprising 9-oxoacridine-10-acetic acid and/or its acceptable salts and/or its esters in therapeutically effective amount and chemotherapeutic agent, is selected from the group including complex platinum compounds (in particular, cisplatin, carboplatin, oxalyplatin); antimetabolites (in particular, methotrexate, 5-fluorouracil, fluorafur, 6-mercaptopurin, altretamine, gemcitabine); alkylating agents (in particular, cyclophosphamide, chlorambucil, melphalan); antitumor antibiotics (in particular, doxorubicin, epirubicin, mitoxantrone); taxans (in particular, paclitaxel, docetaxel); topoisomerase I inhibitors (in particular, topotecan, irinotecan).

It shall be also appreciated that each of the above inventive compositions and medicaments may alternatively include, comprise, or be substantially composed of any suitable components disclosed in the present specification, and such compositions and medicaments, including pharmaceutical compositions, and a kit according to the invention, may additionally or alternatively be prepared in such a way that a component, a material, an ingredient or an object could be excluded therefrom, which was used in a corresponding medicament or composition known in the prior art, or which is not necessary to achieve the technical effect of the present invention.

The same refers to method of treatment according to the invention, which alternatively may include, comprise, or be substantially composed of any matching stages disclosed in the present specification, and such inventive methods may additionally or alternatively exclude some steps or objects, which is used in a method, known in the prior art, or which is not necessary to achieve the technical effect of the present invention.

Further, the invention is illustrated by specific examples not limiting the scope of the present invention.

Materials and Methods

Commercially available preparations of CMA salts, for example sodium CMA salt (preparation Neovir®, Pharmsynthez, Russia), meglumine CMA salt (preparation Cycloferon®, NTFF Polysan, Russia), salt of CMA with 3-O—(N,N-dimethylamino-n-propyl)-1,2:5,6-di-O-isopropyliden-α,D-glucofuranose (preparation Anandin®, Mediter, Russia) as well as commercially available CMA (Sigma, USA, cat. #17927, catalogue of year 2005), among others, were used in the experiments and clinical studies carried out by the present inventors.

Some other CMA salts as well as esters were synthesized by known, relatively simple methods (see for example: Inglot A. D. et al., Archivum Immunologiae et Therapiae Experimentalis, 1985, vol. 33, pp. 275-285; RU 2135474; RU 2036198; RU 2033413). The protonated form of CMA, as well as CMA esters were dissolved in dimethylsufoxide (DMSO) before addition to a cultural medium in in virto experiments.

A kit for ovarian cancer treatment, comprising 9-oxoacridine-10-acetic acid and/or its salts and/or its esters in a therapeutically effective amount and a chemotherapeutic agent, can be prepared according to the present invention, for example, in the following procedure, including the steps of:

(a) preparation of unit dosage forms (ampoules and/or suppositories and/or tablets and/or capsules) of CMA and/or its acceptable salts and/or its esters;

(b) preparation of unit dosage forms of a chemotherapeutic agent;

(c) packaging the obtained unit dosage forms (vials, ampoules etc.) prepared according to steps (a) and (b) into polyethylene terephthalate (PET) blisters.

(d) packaging the blisters prepared in step (a) into one individual carton box together with the leaflet containing patient information, such as instruction and administration doses.

In some cases, suppositories containing CMA or its pharmaceutically acceptable salt or ester were prepared on the basis of widely used for this purpose suppository masses, such as Witepsol (Witepsol W 35, E 75, Condea Chemie GmbH), in a manner illustrated by the presented examples for for rectal or vaginal administration in clinic.

The experiments to study the CMA influence on the level of active NF κB in ovarian cancer cells were performed using primary cultures of human cancer cells isolated from ascitic fluid of patients with stage of III ovarian cancer verified morphologically.

For this purpose, ascetic fluid was centrifuged at 1000 g for 10 min at 22° C. and cells were twice washed with Eagle's medium. Then the cells were resusupended and cultivated in Dulbecco's modified Eagle's medium which was supplemented with 4 mM L-glutamine, 1.5 g/L of sodium bicarbonate, 4.5 g/L of glucose, 10% of calf serum, 100 IU/ml of penicillin sodium salt, and 100 mkg/ml streptomycin sulfate CO₂ at 37° C. with 5% in air. All below described experiments were performed with cells of passages 6-8.

The level of active NF κB in cell extract was measured with StressXpress NF κB, p50 ELISA Kit (StressGen, cat. No EKS-445) and luminescent spectrophotometer SM 2203 (Solar, Belarus). The changes of NF κB activity were presented as percents of basal (control) NF κB activity, i.e. of NF κB activity out of any influences.

Further experiments were performed to study influence CMA and chemotherapeutic agents on proliferative activity and NF κB activity using human ovarian cancer cell lines OVCAR-3 (ATCC number HTB-161) and SK-OV-3 (ATCC number HTB-77).

The commercially available chemical and pharmaceutical preparations of anti-tumor and hormonal agents were used.

The ability of CMA, its salts and its ester alone or in their combination with a chemotherapeutic and/or a hormonal agent to inhibit proliferation of cancer cells was estimated by measurement of amount of bromodeoxyuridine (BrdU) incorporated into nuclear DNA during incubation.

For this purpose, the cells were incubated with abovementioned investigative agents or their combinations for 24 hours. In separate experiments the cells were incubated with CMA for 12 hours, washed and than exposed to the chemotherapeutic agents for next 12 hours.

Then BrdU was added to the cultivation medium to 10 mkM and cells were incubated 24 hours. Then cells were fixed and incubated with antibodies to BrdU for 2 hors at room temperature.

After addition of tetramethylbenzidine (TMB) an intensity of appeared color was measured with spectrophotometer. The intensity of the color is correlated directly with the amount of newly synthesized DNA. All results on estimation of proliferative response were presented as percent of inhibition of DNA synthesis as compared with control cells (control cells were cells which did not undergo any agents' exposure; and BrdU incorporation level in these cells was taken as 100%). To estimate other possible mechanism of CMA effect on sensitivity of cancer cells to the chemotherapy the interferon level in culture medium was measured with the biologic method. The measurement of interferon level was carried out by titration of the samples on monolayer culture of mice cells L-292 (ATCC number CCL-1). Vesicular stomatitis virus (strain Idiana) was used as the test-virus.

L-929 cells (2×10⁵-3×10⁵/ml) were grown in Eagle's medium supplemented with 10% embryonic calf serum in 96-wells plates at 37° C. under 5% CO₂ to form a confluent monolayer (24-28 hrs). Before the titration the culture medium was removed, and dilutions of mice interferon reference-standard (PBL Biomedical Laboratories, Piscataway, N.J.) (or samples to be tested) prepared in Eagle's medium were added into the wells. Not less than four wells were used for each dilution of each sample. The cells were additionally incubated for 24 hours, and then, the culture medium was removed and the test virus at 100% cytophatic dose was added to the cells. The titer of interferon (in activity units) in each sample was determined as the reciprocal of the dilution of the mixture showing a 50% reduction in cytopathic effect.

The invention will be further described by way of non-limiting example embodiments using the following forms of CMA, its salts and esters according to the invention: CMA (CMA-OH), sodium CMA salt (NaCMA), meglumine CMA salt (MegCMA), eglumine CMA salt (EgCMA), salt of CMA with 3-O—(N,N-dimethylamino-n-propyl)-1,2:5,6-di-O-isopropyliden-α,D-glucofuranose (AminopropylCMA), ethyl ester (EtCMA) and propyl ester (PropCMA).

### Example 1

### The Influence of CMA, Its Salts and Its Esters on the Level of Active NF κB in Human Cancer Cell Line

Human ovarian cancer cells, obtained as it was described above, were incubated in presence of different concentration (from 1×10⁻¹⁰ [symbol] 1×10⁻¹³ M) of CMA, its salts and its esters. CMA (CMA-OH), sodium CMA salt (NaCMA), meglumine CMA salt (MegCMA), eglumine CMA salt (EgCMA), salt of CMA with 3-O—(N,N-dimethylamino-n-propyl)-1,2:5,6-di-O-isopropyliden-α,D-glucofuranose (AminopropylCMA), ethyl ester (EtCMA) and propyl ester (PropCMA) of CMA were used in experiments. After incubation for 8 hours the cells were separated from culture medium by centrifugation. Interferon level was measured in cultural medium. Level of active NF κB was estimated in cell nuclear extract. Interferon level in culture medium was presented in activity units (AU) per ml. The results of measurement of NF κB activity were expressed in percents of control values (the cells incubated without any agents). The results of the experiments are presented in Table 1.

TABLE NO. 1
Decrease of level of active NF κB in cancer
cells under the influence of CMA.

| | Concentrations | Patient 1 | Patient 2 | Patient 3 |
|---|---|---|---|---|
| Control | 0 | 100%/0 | 100%/0 | 100%/0 |
| CMA-OH | 1 × 10⁻¹³ M | 81%/0 | 94%/0 | 85%/ 0 |
| | 1 × 10⁻¹² M | 64%/10 | 59%/20 | 54%/20 |
| | 1 × 10⁻¹⁰ M | 32%/10 | 21%/20 | 33%/20 |
| NaCMA | 1 × 10⁻¹³ M | 85%/0 | 91%/0 | 85%/0 |
| | 1 × 10⁻¹² M | 67%/10 | 52%/20 | 49%/20 |
| | 1 × 10⁻¹⁰ M | 25%/10 | 22%/20 | 28%/20 |
| MegCMA | 1 × 10⁻¹³ M | 80%/0 | 93%/0 | 81%/0 |
| | 1 × 10⁻¹² M | 57%/10 | 54%/20 | 42%/20 |
| | 1 × 10⁻¹⁰ M | 21%/10 | 20%/20 | 25%/20 |
| EgCMA [symbol] | 1 × 10⁻¹³ M | 80%/0 | 98%/0 | 94%/0 |
| | 1 × 10⁻¹² M | 67%/10 | 50%/20 | 71%/20 |
| | 1 × 10⁻¹⁰ M | 22%/10 | 29%/20 | 32%/20 |
| AminopropylCMA | 1 × 10⁻¹³ M | 90%/0 | 94%/0 | 92%/0 |
| | 1 × 10⁻¹² M | 53%/10 | 52%/20 | 50%/20 |
| | 1 × 10⁻¹⁰ M | 18%/10 | 22%/20 | 42%/20 |
| EtCMA | 1 × 10⁻¹³ M | 83%/0 | 89%/0 | 82%/0 |
| | 1 × 10⁻¹² M | 49%/10 | 46%/20 | 55%/20 |
| | 1 × 10⁻¹⁰ M | 32%/10 | 33%/20 | 30%/20 |
| PropCMA | 1 × 10⁻¹³ M | 89%/0 | 91%/0 | 81%/0 |
| | 1 × 10⁻¹² M | 68%/10 | 59%/20 | 64%/20 |
| | 1 × 10⁻¹⁰ M | 32%/10 | 31%/20 | 36%/20 |

The nominator represents the active NF κB (in percents of control), the denominator represents interferon level in the culture medium (in Activity Units (AU) per ml).

From the data presented in the Table No. 2 it is evident that CMA, its salts and esters have direct dose-dependent suppressive influence on level of active NF κB. At the same time induction of interferon is hardly observed or no observed, with no relationship between level of interferon induction and the decrease of active NF κB level. Thus, CMA, its salts and its esters are effective inhibitors of NF κB activity and this ability has no relation to interferon-inducing properties of these compounds

### Example 2

### Effect of CMA, Its Salts and Its Esters on the Level of NF κB Activation Caused by Exposure to Chemotherapeutic Agent

The experiments were performed as described in Example 1. The human ovarian carcinoma cell lines OVCAR-3 was used. The cells were incubated at 37° C. in RPMI 1640 medium supplemented with 10% embryonic calf serum. Complex platinum compounds (cisplatin and others), anthracyclines (doxorubicin and others), antimetabolites (5-fluorouracil (5-FU) and others) and alkylating agents (cyclophosphamide and others). Corresponding chemical agent or the vehicle (control) was added to the culture medium simultaneously with CMA, its salt or ester (1×10⁻¹² M). The cells were separated by centrifugation. The level of active NF κB was assessed in nuclear lysate. The results of measurement NF κB activity were expressed in percents of control values (the cells incubated in the absence of any agents). The results of the experiments are presented in the Table No. 2.

TABLE NO. 2
Effect of CMA, its salts and its esters on the level of NF κB activation caused
by exposure to chemotherapeutic agent.

| CMA, its salt or | | | | | |
|---|---|---|---|---|---|
| ester | | | | | Cyclo- |
| 1 × 10⁻¹² M | Vechicle | Cisplatin | Doxorubicin | 5-FU | phosphamide |
| CMA-OH | 76% | 125%/60% | 540%/200% | 156%/112% | 214%/112% |
| NaCMA | 75% | 122%/64% | 531%/188% | 175%/100% | 210%/111% |
| MegCMA | 88% | 117%/55% | 524%/196% | 160%/99% | 221%/99% |
| EgCMA [symbol] | 89% | 121%/63% | 538%/188% | 171%/88% | 217%/89% |
| AminopropylCMA | 88% | 120%/59% | 550%/210% | 149%/79% | 221%/92% |
| EtCMA | 84% | 119%/66% | 497%/210% | 155%/110% | 210%/87% |
| PropCMA | 84% | 118%/66% | 562%/178% | 148%/86% | 214%/95% |

The nominator represents the level of active (as percents of control) NF κB in the absence of CMA, the denominator represents the level of active (as percents of control) NF κB in the presence of CMA.

From the data presented in the Table No. 2 it is evident that CMA prevents NF κB activation caused by chemotherapeutic agents.

### Example 3

### Effect of CMA, Its Salts and Esters on the Proliferative Activity of Human Ovarian cancer Cells Under Exposure to the Chemotherapeutic Agents of Various Classes and Their Combination

The cells of human ovarian carcinoma cell line SK-OV-3 were incubated for 24 hours in the presence of the chemotherapeutic agent of various classes (or in their combination) and CMA (1×10⁻¹³ M). Then the proliferative activity was determined with BrdU as described above. The results the measurement of proliferative activity was presented as percents of the control values (the cells incubated in the absence of any agents). The results of the experiments are presented in the Table No 3.

TABLE NO. 3
Effect of CMA, its salts and esters on the proliferative activity of human ovarian cancer
cells under exposure to the chemotherapeutic agents of various classes and their
combination.

| CMA or its salt | | | | | Cyclo- | |
|---|---|---|---|---|---|---|
| or ester, | | Cispaltin | Doxorubicin | Paclitaxel | phosphamid | |
| 1 × 10⁻¹³ M | Vechicle | (CP) | (Dox) | (Pcl) | (CF) | CP + Pcl |
| Concentration | — | 2 | 1 | 200 | | 1 and 100, |
| (mkg/ml) | | | | | | correspondingly. |
| CMA-OH | 96 | 60/23 | 48/19 | 56/13 | 64/38 | 32/10 |
| NaCMA | 95 | 52/24 | 52/16 | 49/14 | 72/26 | 34/11 |
| MegCMA | 99 | 64/22 | 43/22 | 58/11 | 63/21 | 36/9 |
| EgCMA [symbol] | 92 | 63/19 | 65/24 | 62/16 | 63/28 | 32/10 |
| AminopropylCMA | 100 | 69/27 | 65/18 | 61/21 | 55/48 | 30/12 |
| EtCMA | 100 | 71/24 | 52/17 | 59/19 | 62/22 | 33/8 |
| PropCMA | 91 | 69/25 | 55/18 | 54/14 | 64/22 | 32/9 |

The nominator represents the proliferative activity level in the absence of CMA, the denominator represents the proliferative activity level in the presence of CMA.

From the data presented in the Table No 3 it is evident that CMA causes marked enhancement of human ovarian cancer cell sensitivity to antiproliferative action of chemotherapeutic agents and their combination. At the same time, CMA itself did not influence on proliferative activity of tumor cells.

### Example 4

### Effect of CMA, Its Salts and Esters on the Proliferative Activity of Human Ovarian Cancer Cells Under Exposure to Chemotherapeutic Agent and Their Combination

The cells of human ovarian carcinoma cell line SK-OV-3 were incubated for 4 hours in the presence of CMA (1×10⁻¹² M) or the vehicle (control). Then the cells were washed three times with fresh medium and incubated for 24 hours in the presence of the chemotherapeutic agent of various classes (or in their combination) (1×10⁻¹² M) or with the vehicle (control). Then the proliferative activity was determined with BrdU as described above. The results the measurement of proliferative activity was presented as percents of the control values (the cells incubated in the absence of any agents). The results of the experiments are presented in the Table No. 4.

TABLE NO 4
Effect of CMA, its salts and esters on the proliferative activity of human ovarian cancer
cells under exposure to chemotherapeutic agent and their combination.

| CMA or its salt or | | Carboplatin | Epirubicin | Docetaxel | |
|---|---|---|---|---|---|
| ester, 1 × 10⁻¹² M | Vechicle | (CP) | (Epi) | (Dcl) | CP + Epi |
| Concentration | — | 1.5 | 1.2 | 200 | 0.80 and |
| (mkg/ml) | | | | | 120, |
| | | | | | correspondingly |
| CMA-OH | 96 | 54/32 | 53/24 | 62/16 | 46/14 |
| NaCMA | 95 | 61/36 | 55/20 | 54/15 | 42/12 |
| MegCMA | 99 | 78/44 | 52/26 | 62/12 | 38/14 |
| EgCMA [symbol] | 92 | 58/30 | 59/18 | 58/18 | 43/12 |
| AminopropylCMA | 100 | 67/31 | 56/19 | 60/18 | 32/16 |
| EtCMA | 100 | 72/31 | 52/27 | 60/23 | 34/18 |
| PropCMA | 98 | 63/27 | 58/16 | 55/14 | 36/12 |

The nominator represents the proliferative activity level after exposure to chemotherapeutic agents without preliminary incubation with CMA, the denominator represents the same but with preliminary incubation with CMA.

From the data presented in Table 3 it is evident that preliminary incubation with CMA causes marked enhancement of human ovarian cancer cell sensitivity to following antiproliferative action of chemotherapeutic agents and their combination. At the same time, CMA itself did not influence on proliferative activity of the tumor cells.

### Example 5

### Effect of CMA, Its Salts and Esters on the Proliferative Activity of Human Ovarian Cancer Cells Under Exposure to the Chemotherapeutic and Hormonoterapeutic Agents of Various Classes and Their Combination

The cells of human ovarian carcinoma cell line SK-OV-3 were incubated for 24 hours in the presence of the chemotherapeutic and hormonoterapeutic agents of various classes (or in their combination) and CMA (1×10⁻¹³ M). Then the proliferative activity was determined with BrdU as described above. The results of the measurement of proliferative activity was presented as percents of the control values (the cells incubated in the absence of any agents). The results of the experiments are presented in the Table No. 5.

TABLE NO. 5
Effect of CMA, its salts and esters on the proliferative activity of human ovarian cancer
cells under exposure to the chemotherapeutic and hormonoterapeutic agents of various
classes and their combination.

| CMA or its | | | | | | |
|---|---|---|---|---|---|---|
| salt or ester | | Cisplatin | Doxorubicin | Paclitaxel | | |
| 1 × 10⁻¹³ M | Vechicle | (CP) | (Dox) | (Pcl) | CP + Toremifen | Pcl + Letrozoel |
| Concentration | — | 2 mkg/ml | 1 mkg/ml | 200 mkg/ml | 2 mkg/ml | 100 mkg/ml |
| | | | | | and 10 nM/L, | and 8 nM/L, |
| | | | | | correspondingly | correspondingly. |
| CMA-OH | 100 | 64/21 | 52/18 | 54/23 | 67/14 | 56/16 |
| NaCMA | 100 | 73/28 | 51/17 | 59/26 | 70/20 | 62/18 |
| MegCMA | 99 | 69/21 | 53/25 | 57/18 | 67/15 | 60/10 |
| EgCMA [symbol] | 98 | 67/28 | 66/25 | 64/26 | 65/17 | 66/14 |
| Aminopropyl | 101 | 73/22 | 62/19 | 68/24 | 74/10 | 69/11 |
| CMA | | | | | | |
| EtCMA | 100 | 76/34 | 54/18 | 61/22 | 79/14 | 72/12 |
| PropCMA | 99 | 71/22 | 52/19 | 64/18 | 68/15 | 68/10 |

The nominator represents the proliferative activity level in the absence of CMA, the denominator represents the proliferative activity level in the presence of CMA.

From the data presented in Table 5 it is evident that CMA causes marked enhancement of human ovarian cancer cell sensitivity to antiproliferative action of chemotherapeutic agents and their combination. The addition of the preparations decreasing estrogen action leads to significant enhancement of this property of CMA. At the same time, CMA itself did not influence on proliferative activity of tumor cells.

### Example 6

### Clinical Efficacy of CMA as a Component of Combined Therapy of Ovarian Cancer

18 ovarian cancer (FIGO stage III) patients with morphologically verified diagnosis were treated with the investigative method. The patients were divided into two groups before the start of the therapy. All patient were treated with 6 courses of the following combined therapy: the taxan (doxitaxel, 75 mg/m², intravenously for 1 hour, once every 3 weeks) and the complex platinum compound (carboplatin, 2 hrs infusion at dose AUC 5 (AUC is the area under curve “concentration of the preparation in the blood/time after start of the infusion”, once every 3 weeks). To calculate the carboplatin dose Calvert formula was used: carboplatin dose (mg)=target AUC(mg/ml/min)×(glomerular filtration rate+25) (ml/min). The glomerular filtration rate was assumed as creatinin clearance value in the patient. Patients in the second group were additionally intravenous treated with CMA sodium salt as 12.5% sterile solution at dose 14 mg/kg once every 3 days during all treatment course. Overall response rate (all cases of complete and partial responses) to the therapy was 55.6% (5 of 9 patients) in the first group and only 77.8% (7 of 9 [symbol] ) in the first group. Thus, addition CMA to scheme of the combined therapy of ovarian cancer lead to the marked increase of clinical efficacy of the chemotherapy and the inventive method improves the clinical outcomes.

### Example 7

### Clinical Efficacy of CMA as a Component of Combined Chemohormone Therapy of Ovarian Cancer Refractory (or Resistant) to First-Line Chemotherapy

55-years old patient with ovarian cancer (stage III) received 2 courses of chemotherapy with a complex platinum compound (cisplatin) given as 2-hours infusion, once every 3 weeks. No therapy effects were observed. Then patient was treated with the variant of the inventive method of treatment. For this purpose the patient was treated with 400 mg/m2 carboplatin, once every 3 week as intravenous infusion, and, also, with antiestrogen toremifen at dose of 60 mg/day per os for first month of the therapy course and then at dose of 80 mg/day starting the second months until the therapy course (15 weeks) was complete. During the treatment course (starting on the first day of the therapy) the patients was additionally treated intramuscularly with CMA, 500 mg/day, once every second day (as sterile solution of meglumine CMA salt (preparation Cycloferon, Polysan, Russia). After the treatment course was complete the partial remission was observed, and the remission was confirmed by the repeated examination in 2 month. Thus, the inventive method is markedly effective against ovarian cancer refractory (or resistant) to first-line chemotherapy.

### Example 8

### Clinical Efficacy of CMA as a Component of Combined Chemohormone Therapy of Ovarian Cancer

68-years old patient with ovarian cancer (stage IV) received 7 courses of combined chemotherapy with the taxan and the complex platinum compound: paclitaxel (175 mg/m²) was given as 3-hours intravenous infusion once every 3 weeks, and cisplatin (100 mg/m²) was given as 2-hours intravenous infusion once every 3 weeks. The diseases stabilization was observed but 7 months later the signs of the disease progression appeared; serum level of CA-125 (a biochemical marker the level of which is correlated with the tumor growth) elevates from 15 up to 35 U/ml. Then patient was treated with the variant of the inventive method of treatment. For this purpose the patients was treated with second-line combined chemotherapy (liposomal doxorubicin, Caelyx, Shering-Plough, USA) given at a dose of 25 mg/m² on day 1 and gemcitabine given at a dose 800 MT/M² on day 1 and day 8 every three weeks). Additionally, during the chemotherapy course, the patient was treated twice daily with suppositories containing 250 mg of CMA and with the preparation blocked estrogen action (oral tamoxifen at dose 40 mg/day). After the treatment course was complete (9 months later) the partial stabilization of the diseases was observed; it is was confirmed by the repeated examination in 2 month; the level of CA125 decreased to 10 U/ml. Thus, the variant of the inventive method as “second-line treatment” is markedly effective against ovarian cancer.

### Example 9

### Clinical Efficacy of CMA as a Component of Combined Second-Line Chemotherapy of Ovarian Cancer

64-old patient with relapse of ovarian cancer (after previous successful therapy with complex platinum compound (cisplatin)) was treated according to the inventive method with 4 course of chemotherapy with a topoisomerase I inhibitor topotecan (Hycamtin, Smith Kline Beecham, UK) at dose of 1.5 mg/m^{2.} Every course represented as 30-min intravenous infusions performed once day for 5 consecutive days. The intervals between courses were 21 days. At the same time, the patient was treated with 100 mg/kg CMA as CMA sodium salt solution (preparartion Neovir, Pharmsynthez, Russia) dissolved in 100 ml of sterile physiological saline solution for dropwise intravenous infusion. Each CMA infusion was carried out before each topotecan infusion. The patients was also administered (between courses of infusion therapy) with intramuscular injection CMA (as CMA meglumine salt (preparation Cycloferon, Polysan, Russia)) in a dose of 10 mg/kg once a day on each second day. The complete remission was observed after forth course. Thus, the inventive method is markedly effective (in particular, as combination of CMA with an topoisomerase I inhibitor) as “second-line treatment” against ovarian cancer relapse followed by remission induced by platinum compound therapy.

### Example 10

### The Kit for Ovarian Cancer Treatment Comprising CMA and the Chemotherapeutic Agent

The kit for ovarian cancer treatment comprising sodium salt of 9-oxoacridine-10-acetic acid and chemotherapeutic agent (from the group of complex platinum compounds) carboplatin, is produced in the following manner:

a) The final formulation (the solution for parenteral use) of sodium salt of 9-oxoacridine-10-acetic acid was prepared. For this, 2.5 g of sodium citrate was dissolved in 900 ml of water for injection; and then, 125 g of powder of sodium salt of 9-oxoacridine-10-acetic acid (Pharmsynthez, Russia) was dissolved in the solution. Then, citric acid was added to adjust pH to 7.8. The prepared solution was filtered sterile through 0.22 mkm membrane filter. The volume of the solution was brought up to 1000 ml with sterile water for injection. The prepared solution was bottled into vials made from brown glass (type I according to European Pharmacopoeia). The vials were sterile corked and sealed with aluminium caps. As a result, the sterile vials, each containing 10 ml of 12.5% solution of sodium salt of 9-oxoacridine-10-acetic acid, were produced.

b) The final formulation of carboplatin (i.e. (cis-diamino-1,1-cyclobutane dicarboxylato platinum II) was produced. For this, 1000 mg of mannitol (SPI Pharma, USA) was added to 1000 mg of carboplatin powder (Yunnan Gejiu Biochemical Pharmaceutical Factory, China), then 100 ml of water for injection was added to the mixture. The mixture was stirring until the components were dissolved completely. The prepared solution was filtered sterile through 0.22 mkm membrane filter and bottled into sterile glass vials. The vials were sterile corked and sealed with aluminium caps. As a result, the sterile 10 ml vials, each containing 100 mg of carboplatin, were produced.

c) The prepared vials with solution of sodium salt of 9-oxoacridine-10-acetic acid were placed into polyethylene terephthalate (PET) blisters (5 vials per blister).

d) The prepared vial with carboplatin solution were placed into polyethylene terephthalate (PET) blisters as well (5 vials per blister).

e) 2 prepared blisters with the vials with solution of sodium salt of 9-oxoacridine-10-acetic acid and 1 prepared blister with the vials with carboplatine solution placed into individual carton box (together with the patient information leaflet).

### Example 11

### The Study of the Ability of CMA Administered in vivo in Interferon-Inducing Doses as Well at Doses Exceeding the Maximal Interferon Inducing Doses to Enhance the Action of a Chemotherapeutic Agent on the NF κB Activity in Tumor Cell as Well on Survival Rate of Tumor-Bearing Animals

To study the ability of different amounts of CMA to inhibit the NF κB activity, tumor growth and to increase the survival rate in combination with a chemotherapeutic agent, BALB/c female nude mice (6 to 8 weeks of age) were intraperitoneally injected with SK-OV-3 cells (1×10⁷ cells/0.5 ml media/mouse). The all mice were divided into the several groups of animals (32 animals per group) which were treated with different doses of CMA, its salts or its ester. All mice were injected also ether with vehicle (diluent) or with chemotherapeutic agent or with or combinations of chemotherapeutic agent. Some groups were injected additionally with one or other aromatase inhibior, along with chemotherapeutic agent. Cisplatin (CP) was administered at dose 5 mg/kg intraperitoneally once weekly for the first 3 weeks; Doxorubicin (Dox) was given 8 mg/kg intravenously on days 0 and 7; Paclitaxel (Pcl) was injected intravenously at dose 10 mg/kg body weight thrice weekly, on alternate days for 4 weeks; 10 mg/kg/day. Toremifen was administered per os at dose 10 mg/kg/day in starch gel. Anastozole was injected subcutaneously with 5 μg of tamoxifen citrate in 0.1 mL of PBS. CMA was administered once a day on each second day.

A ½ mouse in each group was sacrificed 30 days after inoculation of the ovarian carcinoma cells and examined: for the formation of intraperitoneal dissemination as well for level of active the NF κB in the cancer cells of ascetic fluid (the level of active NF κB was determined with StressXpress NF κB, p50 ELISA Kit as described above; these results were showed as percents of control (vehicle-treated mice)). Also, the day of 50% survival rate was scored for other 16 animals in each group group. The serum alpha interferon (alpha IFN) level was measured 2 hours after first administration of CMA, its salts or it ester. Mouse IFN-alpha was measured in serum was detected (in pg/ml) using the enzyme-linked immunosorbent assay kit purchased from PBL Biomedical Laboratories (USA, Piscataway, N.J.).

The results are presented in the Tables Nos. 5, 6, 7.

TABLE NO. 5
The level of inteferon induction, NF κB activity in tumor cell and survival rate of tumor-
bearing animals treated with combination of 9-oxoacridine-10-acetic acid with
chemotherapeutic agents and aromatase inhibitors.

| CMA- | | | | | | |
|---|---|---|---|---|---|---|
| OH dose | | | | | | |
| in mg/kg | | | | | | |
| of body | | Cisplatin | Doxorubicin | Paclitaxel | | Pcl + |
| weight* | Vechicle | (CP) | (Dox) | (Pcl) | CP + Toremifen | Anastozole |
| 0 mg/kg | 5/100/24 | 4/380/34 | 3/450/36 | 8/420/41 | 4/320/38 | 8/380/46 |
| 2 mg/kg | 57/100/26 | 54/390/32 | 68/440/36 | 59/410/42 | 54/340/42 | 60/390/52 |
| 5 mg/kg | 116/100/24 | 120/350/34 | 120/430/36 | 120/380/41 | 137/300/48 | 148/350/54 |
| 10 mg/kg | 156/100/28 | 180/340/34 | 160/380/36 | 180/350/41 | 158/280/51 | 188/330/56 |
| 14 mg/kg | 166/98/26 | 170/150/57 | 148/210/52 | 170/200/54 | 156/150/61 | 170/200/64 |
| 20 mg/kg | 161/92/25 | 167/110/68 | 149/80/71 | 172/110/66 | 155/120/83 | 174/93/73 |
| 100 mg/kg | 148/89/29 | 172/90/76 | 154/72/83 | 165/94/120 | 149/50/98 | 171/180/144 |
| 500 mg/kg | 159/88/29 | 180/84/88 | 146/87/92 | 173/71/135 | 140/47/115 | 182/50/148 |

*Comments: the first cipher presents interferon level (pg/ml, mean), the second presents level of NF kB (as percent from the control, mean), the third presents the day when 50% of animals in the group were still alive

TABLE NO. 6
The level of interferon induction, NF κB activity in tumor cell and survival rate of tumor-
bearing animals treated with a combination of sodium salt of 9-oxoacridine-10-acetic acid
with chemotherapeutic agents and aromatase inhibitors.

| NaCMA, | | | | | | |
|---|---|---|---|---|---|---|
| dose in | | | | | | |
| mg/kg of | | | | | | |
| body | | Cisplatin | Doxorubicin | Paclitaxel | | Pcl + |
| weight* | Vehicle | (CP) | (Dox) | (Pcl) | CP + Toremifen | Anastozole |
| 0 mg/kg | 3/100/26 | 2/356/44 | 4/470/37 | 6/411/40 | 8/310/36 | 9/372/45 |
| 2 mg/kg | 52/100/27 | 48/341/35 | 63/435/39 | 57/390/39 | 49/330/41 | 54/380/48 |
| 5 mg/kg | 112/100/28 | 115/345/38 | 118/421/37 | 127/370/39 | 140/280/50 | 139/340/48 |
| 10 mg/kg | 154/100/31 | 160/333/37 | 172/360/38 | 172/340/38 | 148/270/53 | 176/320/55 |
| 14 mg/kg | 154/96/22 | 160/145/63 | 145/190/54 | 165/193/55 | 152/140/55 | 161/166/77 |
| 20 mg/kg | 155/90/21 | 172/90/70 | 140/73/69 | 166/112/67 | 161/110/83 | 141/98/81 |
| 100 mg/kg | 141/88/23 | 157/85/78 | 178/71/80 | 152/90/118 | 143/46/89 | 168/175/138 |
| 500 mg/kg | 149/78/26 | 177/83/90 | 165/85/100 | 168/70/138 | 144/43/114 | 161/55/149 |

*Comments: the first cipher presents interferon level (pg/ml, mean), the second presents level of NF kB (as percent from the control, mean), the third presents the day when 50% of animals in the group were still alive; doses of NaCMA are calculated on the base of 9-oxoacridine-10-acetic acid.

TABLE NO. 7
The level of inteferon induction, NF κB activity in tumor cell and survival rate of tumor-
bearing animals treated with combination of ethyl ester of 9-oxoacridine-10-acetic acid
with chemotherapeutic agents and aromatase inhibitors.

| NaCMA, | | | | | | |
|---|---|---|---|---|---|---|
| dose in | | | | | | |
| mg/kg | | | | | | |
| of body | | Cisplatin | Doxorubicin | Paclitaxel | CP + | Pcl |
| weight* | Vehicle | (CP) | (Dox) | (Pcl) | Toremifen | Anastozole |
| 0 mg/kg | 8/100/27 | 3/420/42 | 5/430/43 | 5/442/38 | 3/580/34 | 2/390/55 |
| 2 mg/kg | 44/100/30 | 44/350/43 | 66/425/47 | 54/350/40 | 44/470/39 | 55/370/58 |
| 5 mg/kg | 130/100/26 | 125/325/41 | 112/420/42 | 122/290/39 | 138/290/51 | 143/340/57 |
| 10 mg/kg | 166/100/27 | 150/310/45 | 162/350/58 | 168/270/42 | 137/220/53 | 182/290/55 |
| 14 mg/kg | 155/96/27 | 149/115/60 | 160/180/64 | 166/150/65 | 151/150/55 | 161/160/97 |
| 20 mg/kg | 166/90/23 | 152/85/72 | 157/83/70 | 165/120/77 | 152/110/86 | 159/100/100 |
| 100 mg/kg | 151/88/25 | 159/80/60 | 162/72/82 | 151/83/110 | 151/49/88 | 160/60/118 |
| 500 mg/kg | 155/78/25 | 148/73/86 | 141/61/96 | 157/65/125 | 157/30/124 | 151/40/131 |

*Comments: the first cipher presents interferon level (pg/ml, mean), the second presents level of NF kB (as percent from the control, mean), the third presents the day when 50% of animals in the group were still alive; doses of EtCMA are calculated on the base of 9-oxoacridine-10-acetic acid.

The data presented in the Tables 5, 6, 7 shows that 9-oxoacridine-10-acetic acid, its salts and esters possess powerful ability to potent the action of chemotherapeutic agents, an to suppress NF κB activity; these features of CMA are not depends on its interferon inducing activity; the effect was observed (and observed more clearly) even CMA was administered in doses greatly exceeded maximal interferon-inducing doses. Addition of aromatase inhibitors to the combination “CMA+chemotherapy” lead to significant synergic enhancement of such treatment.

I claim:

1. A method of treating ovarian cancer in a subject in need of such a treatment, the method comprising administering 9-oxoacridine-10-acetic acid and/or salts and/or esters thereof in a combination therapy with one or more chemotherapeutic agents, wherein 9-oxoacridine-10-acetic acid and/or salts and/or esters thereof is administered in amounts effective in potentiating the action of the said chemotherapeutic agent or agents.

2. A method of claim 1, wherein the said salts of 9-oxoacridine-10-acetic acid are selected from sodium, meglumine, eglumine salts and 3-O—(N,N-dimethylamino-n-propyl)-1,2:5,6-di-O-isopropyliden-α,Dglucofuranose salt.

3. A method of claim 1, wherein the said chemotherapeutic agent is selected from complex platinum compound, antimetabolite, alkylating agent, antitumor antibiotic, taxan derivative, topoisomerase I inhibitor.

4. A method of claim 3, wherein the said complex platinum compound is selected from cisplatin, carboplatin, oxaliplatin, methotrexate, 5-fluorouracil, fluorafur, 6-mercaptopurin, altretamine, gemcitabine, cyclophosphamide, chlorambucil, melphalan, doxorubicin, epirubicin, mitoxantrone, paclitaxel, docetaxel, topotecan and irinotecan.

5. A method of claim 1, wherein the therapy further includes hormonotherapy aimed to decreasing the effect of endogenous estrogens.

6. A method of claim 5, wherein the said hormonotherapy includes administration of one or more agents selected from anti-estrogens, progestins, aromatase inhibitors, LHRH-antagonists, LHRH-agonist.

7. A method according to claim 1, wherein a single dose of 9-oxoacridine-10-acetic acid and/or salt and/or ester thereof is about 14 mg/kg to about 100 mg/kg calculated based on 9-oxoacridine-10-acetic acid.

8. A method of treating ovarian cancer in a subject in need of such a treatment, comprising administering 9-oxoacridine-10-acetic acid and/or salt and/or ester thereof in a combination therapy with one or more chemotherapeutic agents, wherein 9-oxoacridine-10-acetic acid and/or salt and/or ester thereof is administered in amounts effective in reducing active NF kB level.

9. A method of claim 8, wherein the said salts of 9-oxoacridine-10-acetic acid are selected from the group including sodium, meglumine, eglumine salts and 3-O—(N,N-dimethylamino-n-propyl)-1,2:5,6-di-O-isopropyliden-α,Dglucofuranose salt.

10. A method of claim 8, wherein the said chemotherapeutic agent is selected from a complex platinum compound, antimetabolite, alkylating agent, antitumor antibiotic, taxan derivative, and topoisomerase I inhibitor.

11. A method of claim 10, wherein the said chemotherapeutic agent is selected from cisplatin, carboplatin, oxaliplatin, methotrexate, 5-fluorouracil, fluorafur, 6-mercaptopurin, altretamine, gemcitabine, cyclophosphamide, chlorambucil, melphalan, doxorubicin, epirubicin, mitoxantrone, paclitaxel and docetaxel, topotecan, irinotecan.

12. A method of claim 8, wherein the combination therapy further includes hormonotherapy aimed at decreasing the effect of endogenous estrogens.

13. A method of claim 8, wherein the said hormonotherapy includes administration of one or more agents selected from anti-estrogens, progestins, aromatase inhibitors, LHRH-antagonists, LHRH-agonist.

14. A method of claim 8, wherein a single dose of 9-oxoacridine-10-acetic acid and/or salt and/or ester thereof is about 14 mg/kg to about 100 mg/kg calculated based on 9-oxoacridine-10-acetic acid.

15. A pharmaceutical kit for treating ovarian cancer in a patient in need of such treatment, the kit comprising a unit dosage of 9-oxoacridine-10-acetic acid and/or salt and/or ester thereof and a unit dosage of a chemotherapeutic agent, wherein the unit dosage of 9-oxoacridine-10-acetic acid and/or salt and/or ester thereof is effective to potentiate the action of the said chemotherapeutic agent.

16. A pharmaceutical kit according to claim 15 wherein the said salt of 9-oxoacridine-10-acetic acid is selected from sodium, meglumine, eglumine salts and 3-O—(N,N-dimethylamino-n-propyl)-1,2:5,6-di-O-isopropyliden-α,D-glucofuranose salt.

17. A pharmaceutical kit according to claim 15, wherein the said chemotherapeutic agent is selected from complex platinum compound, antimetabolite, alkylating agent, antitumor antibiotic, taxan.

18. A pharmaceutical kit according to claim 15, wherein the unit dosage of 9-oxoacridine-10-acetic acid and/or salt and/or ester thereof provides the administration of about 14 mg/kg to about 100 mg/kg.

19. A pharmaceutical kit for treating ovarian cancer in a patient in need of such treatment, the kit comprising a unit dosage of 9-oxoacridine-10-acetic acid and/or salt and/or ester thereof and a unit dosage of a chemotherapeutic agent, wherein 9-oxoacridine-10-acetic acid and/or salt and/or ester thereof is present in amount effective in reducing active NF kB level.

20. A pharmaceutical kit according to claim 19 wherein the said salt of 9-oxoacridine-10-acetic acid is selected from sodium, meglumine, eglumine salts and 3-O—(N,N-dimethylamino-n-propyl)-1,2:5,6-di-O-isopropyliden-α,D-glucofuranose salt.

21. A pharmaceutical kit according to claim 19, wherein the said chemotherapeutic agent is selected from complex platinum compound, antimetabolite, alkylating agent, antitumor antibiotic, taxan.

22. A pharmaceutical kit according to claim 19, wherein the unit dosage of 9-oxoacridine-10-acetic acid and/or salt and/or ester thereof provides the administration of about 14 mg/kg to about 100 mg/kg.
